(19) United States
(12) Patent Application Publication
(10) Pub. No.: US 2011/0008904 A1
(43) Pub. Date: Jan. 13, 2011

(54) METHOD FOR DETERMINING IF A PATIENT HAS A TRAUMATIC BRAIN INJURY AND RELATED APPARATUS

(75) Inventor: JULES B. PUSCHETT, Temple, TX (US)

Correspondence Address: ECKERT SEAMANS CHERIN & MELLOTT, 600 GRANT STREET, 44TH FLOOR, PITTSBURGH, PA, 15219, US

(73) Assignee: SCOTT AND WHITE MEMORIAL HOSPITAL AND SCOTT, SHERWOOD, AND BRINDLEY FOUNDATION, TEMPLE, TX (US)

(21) Appl. No.: 12/781,464

(22) Filed: May 17, 2010

Related U.S. Application Data
(60) Provisional application No. 61/224,526, filed on Jul. 10, 2009.

(51) Int. Cl. G01N 33/50

(52) U.S. Cl. 436/93; 422/68.1

(57) ABSTRACT

A method for determining if a patient has a traumatic brain injury includes obtaining a body specimen from the patient, determining the concentration of marinobufagenin in the body specimen, comparing the concentration of marinobufagenin to the concentration in such body specimens in normal persons, and if the marinobufagenin concentration is substantially above the concentration of a normal person, concluding traumatic brain injury exists. In a preferred embodiment, a substantial elevation is deemed to be an increase of about 30 percent above the marinobufagenin concentration of a normal person. The body specimen may be blood or urine. If a substantial elevation is deemed to exist, the magnitude from the departure from the concentration of a normal person may be employed in determining the timing and nature of treatment provided to the patient. The method may be repeated at predetermined intervals to monitor changes in the marinobufagenin with time. Corresponding apparatus is provided.

### BACKGROUND OF THE INVENTION

1. Field of the Invention

The present invention relates to a method for analyzing a patient's body specimen to determine the concentration of marinobufagenin in the specimen and compare that determination with the concentration of marinobufagenin in normal persons in order to determine if a patient has a traumatic brain injury. The invention also provides diagnostic apparatus, which may be employed to receive the body specimen and make the desired comparison.

2. Description of the Prior Art

It has long been known that traumatic brain injuries can result in temporary problems, permanent problems, and in some instances, death. It has, more recently, been recognized that such brain injuries may not, initially, produce symptoms which cause either the patient, others, and even medically-skilled individuals to be concerned even though, within a day or two, very serious consequences may result.

In one relatively-recent incident, an actress had a minor fall while skiing. She, at the time, felt no adverse consequences and refused medical attention. Several hours later, in her hotel room, she complained of a headache, and within hours, she was in critical condition. She died two days later. As a result of instances, such as this, it is important that there be prompt and effective medical attention to situations, wherein traumatic brain injury may have occurred.

At present, one of the rather subjective and not totally effective diagnostic techniques when traumatic brain injury is suspected involves a number of examining techniques. The patient receives a neurological examination which consists of the following: 1) mental status, 2) motor function, 3) sensory examination, 4) deep tendon reflexes, 5) station, gait, and equilibrium, 6) cranial nerve function. Included in the mental status examination are: a) level of consciousness, b) short and long term memory, c) knowledge of person and place and d) questions about symptoms: headache, dizziness, blurry vision, etc. In addition, the patient may also have radiological studies which could include CT scan of the head, MRI, PET scan. It has been reported that in the early stages of (especially mild) traumatic brain injury, the imaging techniques may not be sufficiently sensitive to detect an abnormality. Furthermore, the patient's cognitive skills may not be impaired initially, and there may be few, if any, symptoms. Patients are often observed over 24-48 hours and are awakened regular intervals (e.g., every 3-4 hours) to assure that they are able to be aroused. Narcotics for headache or other pain are not given, so that their effects do not cloud the issue of the patient's arousal state. A computerized test which determines level of cognition and reaction time is often employed with repetitive examinations.

One of the problems with this approach in diagnosing potential traumatic brain injuries is that it is not one which always provides precise, timely, objective information. It is also subject to individual variations from person-to-person. Further, if the person is asymptomatic at the time, the conclusion might be that there is no problem, and the individual might be encouraged to go back to normal activities. Such guidance could potentially be injurious to the person's health and could even lead to fatal consequences.

In spite of the foregoing, there remains a very real and substantial need for a method of early and effective determination as to whether an individual has suffered a traumatic brain injury and how severe it might be.

### SUMMARY OF THE INVENTION

The present invention has met the above-described need for providing a method of early and accurate determination of whether a patient has suffered traumatic brain injury. The method includes obtaining a body specimen, which, for example, may be blood or urine from the patient, determining the concentration of marinobufagenin in the body specimen, and comparing the concentration of marinobufagenin in the specimen with the marinobufagenin concentration in normal persons. If the marinobufagenin concentration is substantially above the concentration in normal persons, this indicates that the individual has a traumatic brain injury. In a preferred embodiment, the elevation of marinobufagenin, which could lead to the conclusion that a traumatic brain injury has occurred, will involve an increase in concentration over a normal person of at least about 30 percent.

The method may be repeated with the results compared with at least one prior result from testing that patient with that method if such concentration has previously been obtained.

If a substantial elevation in marinobufagenin above that of a normal person exists, the concentration may be employed in determining the timing and nature of the treatment to be provided to the patient.

In another embodiment of the invention, apparatus for determining the presence of a traumatic brain injury in a patient includes a specimen receiver for receiving a patient's body specimen, such as urine or blood. The specimen receiver is structured to employ urine or blood as the body specimen. An analyzer determines the concentration of marinobufagenin in the patient body specimen. This is compared with the concentration to the range in normal persons to determine whether a substantial elevation in marinobufagenin exists in the body specimen obtained from the patient. The presence of a substantial elevation above the normal range is indicative of a traumatic brain injury. In the preferred form, the apparatus may be calibrated to provide an indication that a substantial elevation exists if the elevation of marinobufagenin is at least about 30 percent above the range of normal persons.

It is an object of the present invention to provide a method of promptly and effectively testing an individual by means of a body specimen to determine if the individual has suffered a traumatic brain injury.

It is another object of the present invention to provide such a method wherein blood, either as blood serum or blood plasma, may be employed as the body specimen.

It is a further object of the present invention to provide such a method wherein urine may be employed as the body specimen.

It is a further object of the present invention to provide a quantitative determination of a patient's concentration of marinobufagenin in order to determine if a substantial elevation in marinobufagenin in the body specimen, as compared with normal persons, is present.

It is yet another object of the present invention to provide an effective method for early diagnosis of a traumatic brain injury in order to facilitate prompt medical treatment.

It is yet another object of the present invention to provide a reliable, prompt indication of the presence of a traumatic brain injury if such an injury exists even under some circumstances where the prior art approaches would have led to the conclusion that no such problems exist.

It is still a further object of the present invention to provide apparatus which may advantageously be employed in processing the body specimen and making a determination regarding the amount of marinobufagenin present in the patient's body specimen.

It is a further object of the present invention to provide such apparatus which will affect a comparison between the marinobufagenin content of the body specimen and that of normal persons.

It is yet another object of the present invention to provide such a method which is employable in connection with battlefield injuries, as well as for civilian uses.

It is another object of the present invention to monitor the patient's progress utilizing multiple determinations of marinobufagenin over time.

### DETAILED DESCRIPTION OF THE PREFERRED EMBODIMENTS

As employed herein, the term “traumatic brain injury” shall mean a brain injury resulting from direct or indirect shock load or loads applied to the brain causing it to move rapidly and unnaturally within a patient's skull and shall expressly include, but not be limited to, brain injuries caused by: (a) objects penetrating the skull, such as, bullets, arrows, and other physical objects which pass through the skull and enter the brain, (b) impact loads applied to the head or other portions of the patient's body, (c) explosions, such as might exist in warfare, through impacting of grenades, bombs, and other explosives, which cause substantial tremors in the earth in relatively-close proximity to where an individual is standing, as well as similar tremors created by nonexplosive means, such as vehicular accidents, collapse of buildings and earthquakes, for example.

The results of traumatic brain injury may be of various types, but in each instance, will involve temporary or permanent reduction in the ability of the brain to function and may cause death.

One of the consequences of a traumatic brain injury frequently is the generation of inflammation within the brain as the shock to the brain serves to increase the permeability of the endothelial cells, thereby permitting loss of fluids from the vascular structure into the brain. Such a leakage frequently occurs due to the increased porocity of the blood vessels resulting from the trauma, thereby causing blood serum to leak through the vessels into the brain area. As this builds up, this can generate inflammation of the brain, which may require surgical intervention.

The method of the present invention involves measuring a body specimen which may be urine or blood, such as blood serum or blood plasma.

The method involves determining if a patient has a traumatic brain injury by obtaining a body specimen from the patient, determining the concentration of marinobufagenin in the body specimen, and comparing the concentration of marinobufagenin with the marinobufagenin concentration in a similar body specimen in a normal person. If the marinobufagenin concentration is substantially above the concentration of a normal person, this indicates that a traumatic brain injury exists.

In a preferred embodiment of the present invention, it is determined that a traumatic brain injury exists if the elevation of marinobufagenin is at least about 30 percent over that of a normal person.

The tests may be repeated periodically to determine trends. If the marinobufagenin concentration continues to increase, this reinforces the conclusion that a traumatic brain injury and probably brain cell damage exist. If it decreases, comparison of the concentration with normal persons will facilitate a determination of reduced concern.

The apparatus of the present invention, for determining the presence of a traumatic brain injury in a patient includes a specimen receiver for receiving a patient's body specimen, such as urine or blood. The specimen receiver is structured to employ urine or blood as the body specimen. The apparatus has an analyzer for determining the patient's concentration of marinobufagenin. That patient concentration is compared with the marinobufagenin in normal persons. The apparatus is structured to determine whether a substantial elevation in marinobufagenin exists in the specimen obtained from the patient. The presence of substantial elevation above the normal range is indicative of a traumatic brain injury. In the preferred form, the apparatus may be calibrated to provide an indication that a substantial elevation exists if the elevation of marinobufagenin is at least about 30 percent above the range of normal persons.

Once the determination that a traumatic brain injury exists has been made, the preferred mode of treatment for a particular patient can be determined. The magnitude of increase of marinobufagenin may be employed to influence the timing and nature of the treatment to be provided.

It will be appreciated, therefore, that the present invention provides a method and related apparatus for making a prompt, reliable, and effective determination as to whether an individual is suffering from traumatic brain injury, so as to minimize the risk of an inaccurate diagnosis leading to potentially serious consequences.

Whereas particular embodiments of the present invention have been described herein for purpose of illustration, it will be evident to those skilled in the art that numerous variations of the details may be made without departing from the invention, as set forth in the appended claims.

What is claimed is:

1. A method of determining if a patient has a traumatic brain injury comprising:
obtaining a body specimen from said patient,
determining the concentration of marinobufagenin in said body specimen,
comparing said concentration of marinobufagenin with the marinobufagenin concentration in such a body specimen in normal patients, and
if the marinobufagenin concentration in said patient is substantially above the concentration in normal patients, concluding that traumatic brain injury exists.

2. The method of claim 1 including
determining whether substantial elevation of marinobufagenin exists if said elevation is at least about 30 percent.

3. The method of claim 1 including
employing blood as said body specimen.

4. The method of claim 3 including
employing as said blood a blood component selected from the group consisting of blood serum and blood plasma.

5. The method of claim 3 including
employing urine as said body specimen.

6. The method of claim 1 including
periodically repeating said method on said patient's body specimen, and
comparing the most current marinobufagenin concentration from said repeated test with at least one prior test of said patient's body specimen.

7. The method of claim 1 including
if there is a determination that the patient has substantially elevated marinobufagenin and has a traumatic brain injury, employing the existence of the substantially-elevated marinobufagenin and the magnitude of the elevation in determining the timing and nature of the therapeutic action to be taken.

8. Apparatus for determining if a patient has a traumatic brain injury comprising
a patient body specimen receiver for receiving a body specimen containing marinobufagenin,
said specimen receiver being structured to employ a specimen selected from a group consisting of urine and blood,
a body specimen analyzer for determining the concentration of marinobufagenin in said patient body specimen and comparing it with the range of marinobufagenin in normal persons, and
determining whether a substantial elevation in marinobufagenin exists in said patient's body specimen relative to the normal range of persons with the presence of a substantial elevation of marinobufagenin from the normal range being indicative of the presence of a traumatic brain disorder.

9. The apparatus of claim 8 including
said analyzer being structured to determine that a substantial elevation exists if said elevation in marinobufagenin is at least about 30 percent above the range of normal persons.
